(19) United States
(12) Patent Application Publication
(10) Pub. No.: US 2025/0235201 A1
(43) Pub. Date: Jul. 24, 2025

(54) SLANTED LOCKING FERRULES FOR PERFORMING TENSIONABLE KNOTLESS CARDIAC VALVE PROCEDURES

(71) Applicant: ARTHREX, INC., Naples, FL (US)

(72) Inventors: Andrew Christian PETRY, Naples, FL (US); Peter J. DREYFUSS, Naples, FL (US); Paul DIGIORGI, Fort Myers, FL (US); Daniel T. ENGELMAN, Longmeadow, MA (US); Marc GERDISCH, Zionsville, IN (US)

(21) Appl. No.: 18/419,722

(22) Filed: Jan. 23, 2024

Related U.S. Application Data
(60) Provisional application No. 63/622,948, filed on Jan. 19, 2024.

(51) Int. Cl. A61B 17/04; A61F 2/24

(52) U.S. Cl. CPC A61B 17/0487; A61F 2/24

(57) ABSTRACT

Systems and methods are provided for performing tensionable knotless cardiac valve procedures. A locking ferrule that includes a one-way locking mechanism may be utilized as part of the tensionable knotless cardiac valve procedure for tensioning and locking one or more strands of suture. The locking ferrule may include a slanted distal end for leaning the locking ferrule in a direction away from a structure associated with the heart valve being repaired.

### CROSS-REFERENCE TO RELATED APPLICATIONS

This application claims the benefit of U.S. Provisional Application No. 63/622,948, which was filed on Jan. 19, 2024 and is incorporated hereby by reference in its entirety.

### BACKGROUND

This disclosure relates to the field of surgery, and more particularly to systems and methods for performing cardiac valve procedures.

The human heart includes multiple valves. The valves open and close as the heart pumps to ensure that blood flows in the proper direction. The structure and/or function of a heart valve can become impaired due to various factors such as age, genetic defects, infection, or other chronic conditions, thereby necessitating its surgical repair or replacement.

### SUMMARY

This disclosure relates to systems and methods for performing tensionable knotless cardiac valve procedures. A locking ferrule that includes a one-way locking mechanism may be utilized as part of the tensionable knotless cardiac valve procedure for tensioning and locking one or more strands of suture.

An exemplary locking ferrule for performing a heart valve surgical procedure may include, inter alia, a body that extends along a longitudinal centerline axis between a proximal end and a slanted distal end. The body includes an outer diameter wall, an inner diameter wall, and a cannulation that is circumscribed by the inner diameter wall. A plurality of locking barbs extend into the cannulation and are configured to establish a one-way locking mechanism for lock a suture relative to the body.

An exemplary surgical method may include, inter alia, passing a suture through a tissue of a heart valve, passing the suture through a prosthetic device, shuttling the prosthetic device along the suture and into the heart valve, loading the suture through a cannulation of a locking ferrule, tensioning the suture in a first direction, and locking the suture within the cannulation to prevent movement of the suture in a second direction.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a slanted locking ferrule that includes a one-way locking mechanism.

FIG. 2 is an end view of the locking ferrule of FIG. 1.

FIG. 3 is a cross-sectional view through section 3-3 of FIG. 1.

FIG. 4 is a cross-sectional view through section 4-4 of FIG. 2.

FIG. 5 schematically illustrates loading a suture through a locking ferrule.

FIG. 6 schematically illustrates locking a suture relative to a locking ferrule.

FIGS. 7, 8, 9, 10, and 11 schematically illustrate a surgical method for performing a heart valve repair or replacement.

FIG. 12 illustrates an exemplary inserter device for implanting a locking ferrule relative to a prosthetic device when performing a heart valve repair or replacement procedure.

### DETAILED DESCRIPTION

This disclosure relates to systems and methods for performing tensionable knotless cardiac valve procedures. A locking ferrule that includes a one-way locking mechanism may be utilized as part of the tensionable knotless cardiac valve procedure for tensioning and locking one or more strands of suture. These and other features of this disclosure are described in further detail below.

An exemplary locking ferrule for performing a heart valve surgical procedure may include, inter alia, a body that extends along a longitudinal centerline axis between a proximal end and a slanted distal end. The body includes an outer diameter wall, an inner diameter wall, and a cannulation that is circumscribed by the inner diameter wall. A plurality of locking barbs extend into the cannulation and are configured to establish a one-way locking mechanism for lock a suture relative to the body.

In any further embodiment, the plurality of locking barbs are integral features of the body.

In any further embodiment, each of the plurality of locking barbs includes a pointed tip.

In any further embodiment, each of the plurality of locking barbs is angled in a direction toward the proximal end.

In any further embodiment, the plurality of locking barbs each protrude inwardly from the inner diameter wall.

In any further embodiment, the plurality of locking barbs are arranged in at least a first row and a second row.

In any further embodiment, a first portion of the plurality of locking barbs of the first row are staggered relative to a second portion of the plurality of locking barbs of the second row.

In any further embodiment, the slanted distal end extends along an axis that is transverse to the longitudinal centerline axis.

In any further embodiment, the slanted distal end includes a slanted edge configured for directionally leaning the body away from a structure associated with the heart valve.

In any further embodiment, the slanted edge includes a curved lip.

An exemplary surgical method may include, inter alia, passing a suture through a tissue of a heart valve, passing the suture through a prosthetic device, shuttling the prosthetic device along the suture and into the heart valve, loading the suture through a cannulation of a locking ferrule, tensioning the suture in a first direction, and locking the suture within the cannulation to prevent movement of the suture in a second direction.

In any further embodiment, a locking barb of the locking ferrule locks the suture relative to a body of the locking ferrule.

In any further embodiment, the locking barb protrudes inwardly from an inner diameter wall of the locking ferrule.

In any further embodiment, the locking barb engages a thickened section of the suture to prevent its movement in the second direction.

In any further embodiment, loading the suture through the cannulation includes pulling the suture through the cannulation in the first direction with a suture loader.

In any further embodiment, the method includes moving an eyelet of the suture loader in the second direction through the cannulation prior to pulling the suture through the cannulation.

In any further embodiment, pulling the suture through the cannulation includes pulling a thinned section of the suture through the cannulation.

In any further embodiment, the method includes, prior to tensioning the suture, leaning the locking ferrule in a direction away from a structure associated with the heart valve.

In any further embodiment, leaning the locking ferrule includes utilizing an alignment tool of an insertion device as a visual cue for positioning a slanted edge of the locking ferrule relative to the structure.

In any further embodiment, the prosthetic device is an annuloplasty ring or a replacement heart valve.

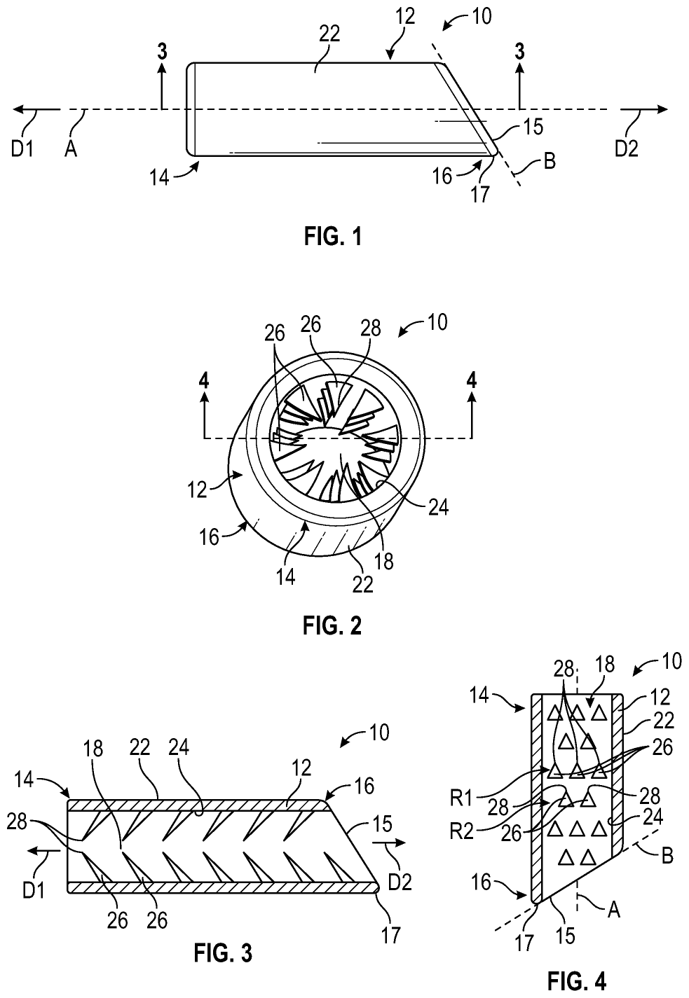

FIGS. 1-6 illustrate an exemplary locking ferrule 10 that can be used when performing tensionable knotless cardiac valve procedures. For example, the locking ferrule 10 could be utilized during surgical methods for repairing or replacing a human heart valve (e.g., aortic, mitral, tricuspid, or pulmonary).

The locking ferrule 10 may include a body 12 that extends along a longitudinal centerline axis A between a proximal end 14 and a distal end 16. The body 12 may be tubular shaped and may be constructed from either metallic materials or plastic materials. However, the specific size, shape, and material make-up of the body 12 are not intended to limit this disclosure.

The distal end 16 of the body 12 may extend along an axis B that is angled relative to the longitudinal centerline axis A. In an embodiment, the axis B extends at a transverse angle relative to the longitudinal centerline axis A. The distal end 16 may thus provide a sloped or slanted edge 15 of the body 12. The slanted edge 15 enables the locking ferrule 10 to be directionally leaned away from the orifice, leaflets, and/or other critical structures of the valve being repaired, for example, thereby reducing the risk of perforation.

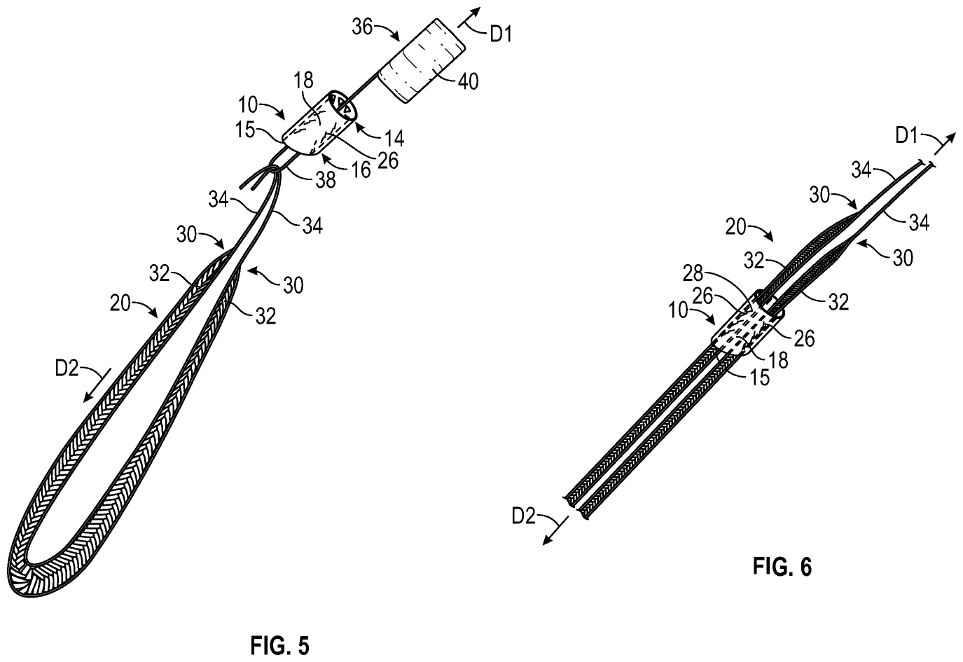

A cannulation 18 may extend through the body 12 and may establish an internal passageway for accommodating one or more strands of suture 20 (see, for example, FIGS. 5 and 6). The cannulation 18 may extend across an entire length of the body 12 and thus extends from the proximal end 14 to the distal end 16. In an embodiment, the longitudinal centerline axis A bisects the cannulation 18.

The body 12 may include an outer diameter wall 22 and an inner diameter wall 24. The outer diameter wall 22 may be smooth or could alternatively include threads, barbs, or other features for facilitating bone fixation. The inner diameter wall 24 may circumscribe the cannulation 18. In some embodiments, the cannulation 18 may taper in a direction toward the distal end 16 and is therefore narrower within the distal end 16 compared to within the proximal end 14.

A plurality of locking barbs 26 may protrude inwardly from the inner diameter wall 24. The locking barbs 26 may therefore occupy at least a portion of the open space of the cannulation 18. In an embodiment, the locking barbs 26 are integrally formed (e.g., molded) features of the body 12 of the locking ferrule 10. The locking barbs 26 may be provided along an entire length of the cannulation 18 or at only select portions thereof. The locking barbs 26 may be either rigid or flexible structures.

The locking barbs 26 may be arranged in multiple rows along the length of the cannulation 18. For example, the locking barbs 26 may be arranged in a least a first row R1 and a second row R2 (see FIG. 4). In an embodiment, the locking barbs 26 of the second row R2 are staggered relative to the locking barbs 26 of the first row R1 (see FIG. 4).

Each locking barb 26 may include a sharp or pointed tip 28, and each locking barb 26 may be angled in a direction toward the proximal end 14. The locking barbs 26 may therefore establish a one-way locking mechanism that permits one or more sutures 20 to pass through the cannulation in a first direction D1 while preventing the suture(s) 20 from being tensioned or otherwise moved in a second direction D2. The staggered relationship of the rows of locking barbs 26 may provide for maximum engagement with the suture 20 one it is passed through the cannulation 18.

The slanted edge 15 of the body 12 may include a curved lip 17. The curved lip 17 provides a relatively smooth surface for suture 20 to glide over as it is being shuttled through the locking ferrule 10. The curved lip 17 may be provided at a distal-most point of the distal end 16, along portions of the outer diameter wall 22 near the distal end 16, or both.

Referring now primarily to FIGS. 5 and 6, with continued reference to FIGS. 1-4, one or more sutures 20 may be passed through the cannulation 18 of the locking ferrule 10. The suture 20 may be FiberWire®, FiberTape®, or any other suitable suture product. FiberWire® and FiberTape® are example suture products marketed and sold by Arthrex, Inc. However, other suture products could be utilized in combination with the locking ferrule 10 within the scope of this disclosure. The size, shape, and type of suture utilized in conjunction with the locking ferrule 10 are not intended to limit this disclosure.

The suture 20 may include a varying thickness. The suture 20 may therefore include one or more tapered regions 30 where the suture 20 transitions between a thickened section 32 and a thinned section 34. In an embodiment, the thickened sections 32 are about twice as thick as the thinned sections 34. However, other ratios (e.g., 1.5:1, 3:1, etc.) between the relative thicknesses (e.g., outer diameters) of the thickened section 32 and the thinned section 34 are contemplated within the scope of this disclosure.

The one or more sutures 20 may be passed through the cannulation 18 of the locking ferrule 10 using a suture loader 36. An eyelet 38 of the suture loader 36 may be passed through the cannulation 18 (e.g., by inserting the eyelet 38 at the proximal end 14 of the body 12 and then moving the suture loader 36 in the second direction D2). One or more thinned sections 34 of the suture 20 may then be loaded through the eyelet 38. The suture loader 36 may then be pulled, via a handle 40, in the first direction D1 to pass the suture 20 through the cannulation 18. The curved lip 17 may help avoid damaging the suture 20 as it is received within and passed through the cannulation 18 of the locking ferrule 10.

Once the suture 20 has been passed through the cannulation 18, the suture 20 may be tensioned in the first direction D1 to lock the suture 20 relative to the locking ferrule 10. The locking barbs 26 prevent the suture 20 from backing up or otherwise moving in the second direction D2. The pointed tips 28 of the locking barbs 26 may interdigitate with one or more of the thickened sections 32 of the suture 20 in order to lock the suture 20 and prevent it from moving in the second direction D2.

Although shown as locking a single folded suture 20 in the above implementations, the locking ferrule 10 could be configured to receive and lock single strand of unfolded suture, multiple strands of suture, and/or sutures of varying shapes and sizes.

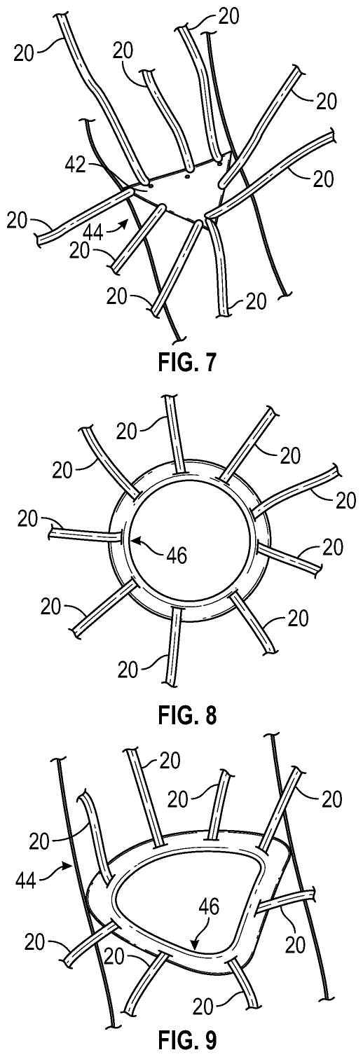

The locking ferrules 10 described above may be utilized to tension and knotlessly fixate one or more sutures 20 as part of various surgical methods. FIGS. 7-11 schematically illustrate one such surgical method for performing a heart valve repair or replacement.

In an embodiment, the surgical method is performed as an arthroscopic procedure by working through various arthroscopic portals. However, the exemplary surgical method could alternatively be performed as an open procedure within the scope of this disclosure.

A plurality of sutures 20 may be passed through a tissue 42 (e.g., muscle) associated with a heart valve 44 that includes a defect (see FIG. 7). The sutures 20 may subsequently be passed through a prosthetic device 46 (see FIG. 8). The prosthetic device 46 may be an annuloplasty ring or a ring portion of a replacement valve, for example. The prosthetic device 46 may next be shuttled down over the sutures 20 to position the prosthetic device 46 at a desired location within the heart valve 44 (see FIG. 9).

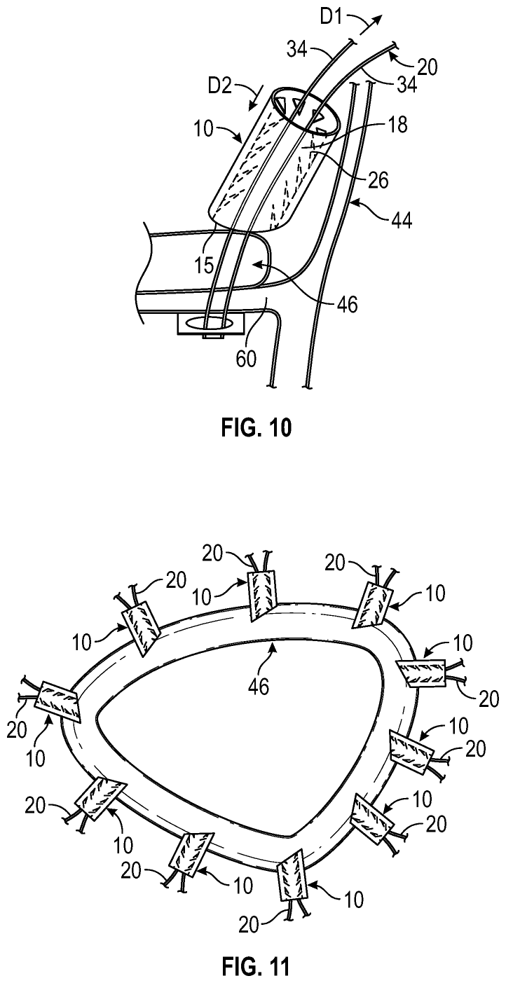

The surgical method may then proceed by shuttling the sutures 20 through a plurality of locking ferrules 10. For example, as shown in FIG. 10, after loading the thinned sections 34 of a first one of sutures 20 through the cannulation 18 of one of the locking ferrules 10 (e.g., in the manner shown in FIGS. 5-6), the suture 20 may be tensioned in the direction D1 to allow the locking ferrule 10 to slide down the suture 20 in the direction D2 and be received in abutting engagement with the prosthetic device 46. Using the slanted edge 15 as a visual guide, the locking ferrule 10 may be directionally leaned away from leaflets 60 or other structures of the heart valve 44 to avoid undesirable perforation as the locking ferrule 10 is shuttled down and into engagement with the prosthetic device 46.

Further tension of the suture 20 in the direction D1 allows the pointed tips 28 of the locking barbs 26 to interdigitate with the thickened sections 32 of the suture 20 to lock the suture 20 and prevent sliding in the second direction D2. This process may be repeated for each previously passed suture 20 in order to knotlessly fixate the prosthetic device 46 within the heart valve 44 using a plurality of the locking ferrules 10 (see FIG. 11). The excess length of the sutures 20 that extend outside of the cannulation 18 of each locking ferrule 10 may be removed (e.g., cut) once a desired level of tensioning and locking has been achieved.

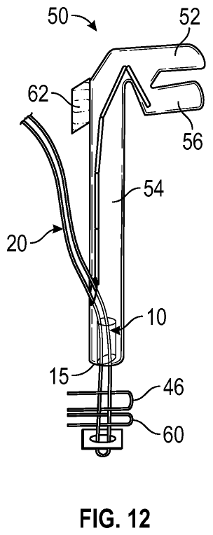

FIG. 12 illustrates an inserter device 50 that can be used to implant the locking ferrule(s) 10 relative to the prosthetic device 46. The inserter device 50 may include a handle 52 and a delivery shaft 54 that extends from the handle 52. One or more locking ferrules 10 may be preloaded into the delivery shaft 54. A trigger 56 of the handle 52 may be squeezed or otherwise actuated to eject the locking ferrules 10 from the delivery shaft 54 one at a time. For example, the trigger 56 may be operably connected with a driver member (not shown) that extends through the delivery shaft 54 and is operable to deploy each of the locking ferrules 10 from the delivery shaft 54 one at a time.

The inserter device 50 may include an alignment tool 62. In an embodiment, the alignment tool 62 is configured as a fin that protrudes outwardly from the delivery shaft 54. However, other configurations are contemplated within the scope of this disclosure. The alignment tool 62 may provide a visual cue for predicting a lean direction of the slanted edge 15 of the locking ferrule 10 relative to the underlying structures (e.g., the prosthetic device 46, the leaflets 60, etc.). The alignment tool 62 can therefore be used to lean the locking ferrule 10 away the underlying structures to avoid undesirable perforation during the repair.

The systems and methods of this disclosure may be utilized to perform tensionable knotless valve repair or replacement procedures. The locking ferrules provide a one-way mechanism for tensioning and retensioning suture(s) at various points of the procedure, including subsequent to implantation of a prosthetic device, thus providing numerous advantages over prior techniques.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A locking ferrule for performing a cardiac valve procedure, comprising:
a body extending along a longitudinal centerline axis between a proximal end and a slanted distal end;
the body including an outer diameter wall, an inner diameter wall, and a cannulation that is circumscribed by the inner diameter wall; and
a plurality of locking barbs extending into the cannulation and configured to establish a one-way locking mechanism for locking a suture relative to the body.

2. The locking ferrule as recited in claim 1, wherein the plurality of locking barbs are integral features of the body.

3. The locking ferrule as recited in claim 1, wherein each of the plurality of locking barbs includes a pointed tip.

4. The locking ferrule as recited in claim 1, each of the plurality of locking barbs is angled in a direction toward the proximal end.

5. The locking ferrule as recited in claim 1, wherein the plurality of locking barbs each protrude inwardly from the inner diameter wall.

6. The locking ferrule as recited in claim 5, wherein the plurality of locking barbs are arranged in at least a first row and a second row.

7. The locking ferrule as recited in claim 6, wherein a first portion of the plurality of locking barbs of the first row are staggered relative to a second portion of the plurality of locking barbs of the second row.

8. The locking ferrule as recited in claim 1, wherein the slanted distal end extends along an axis that is transverse to the longitudinal centerline axis.

9. The locking ferrule as recited in claim 1, wherein the slanted distal end includes a slanted edge configured for directionally leaning the body away from a structure associated with a heart valve being repaired during the cardiac valve procedure.

10. The locking ferrule as recited in claim 9, wherein the slanted edge includes a curved lip.

11. A surgical method, comprising:
passing a suture through a tissue of a heart valve;
passing the suture through a prosthetic device;
shuttling the prosthetic device along the suture and into the heart valve;
loading the suture through a cannulation of a locking ferrule;
tensioning the suture in a first direction; and
locking the suture within the cannulation to prevent movement of the suture in a second direction.

12. The surgical method as recited in claim 11, wherein a locking barb of the locking ferrule locks the suture relative to a body of the locking ferrule.

13. The surgical method as recited in claim 12, wherein the locking barb protrudes inwardly from an inner diameter wall of the locking ferrule.

14. The surgical method as recited in claim 12, wherein the locking barb engages a thickened section of the suture to prevent its movement in the second direction.

15. The surgical method as recited in claim 11, wherein loading the suture through the cannulation includes:
pulling the suture through the cannulation in the first direction with a suture loader.

16. The surgical method as recited in claim 15, comprising:
moving an eyelet of the suture loader in the second direction through the cannulation prior to pulling the suture through the cannulation.

17. The surgical method as recited in claim 15, wherein pulling the suture through the cannulation includes:
pulling a thinned section of the suture through the cannulation.

18. The surgical method as recited in claim 11, comprising, prior to tensioning the suture:
leaning the locking ferrule in a direction away from a structure associated with the heart valve.

19. The surgical method as recited in claim 18, wherein leaning the locking ferrule includes:
utilizing an alignment tool of an insertion device as a visual cue for positioning a slanted edge of the locking ferrule relative to the structure.

20. The surgical method as recited in claim 11, wherein the prosthetic device is an annuloplasty ring or a replacement heart valve.
